(19) United States
(12) Patent Application Publication
(10) Pub. No.: US 2007/0207980 A1
(43) Pub. Date: Sep. 6, 2007

(54) Chp-gemcitabine Combination Agents And Their Use As Anti-Tumor Agents

(76) Inventors: Zoser B. Salama, Berlin (DE); Dagmar Braun, Greifswald-Insel Riems (DE)

Correspondence Address: JOYCE VON NATZMER;PEQUIGNOT + MYERS LLC, 200 Madison Avenue, Suite 1901, New York, NY, 10016, US

(21) Appl. No.: 10/596,409

(22) PCT Filed: Dec. 12, 2004
(86) PCT No.: PCT/DE04/02760

§ 371 Date: Mar. 21, 2007

(30) Foreign Application Priority Data
Dec. 12, 2003 (DE) 103 59 828.6

(51) Int. Cl. A61K 31/7072; A61K 31/4015

(52) U.S. Cl. 514/49; 514/423

(57) ABSTRACT

The invention relates to combined agents comprising cis-hydroxyproline (CHP) and gemcitabine and to the use of said agents in tumor prophylaxis and therapy.

The invention relates to combined agents comprising cis-hydroxyproline (CHP) and gemcitabine and to the use of said agents in tumor prophylaxis and therapy.

Tumors or cancers represent a locally confined increase of tissue volume and thus, in a broader sense, any localized swelling as a result of edemas, acute and/or chronic inflammations, e.g. organ swelling caused by inflammation. More strictly speaking, tumors represent formation of new tissue in the form of a spontaneous, variably disinhibited, autonomous and irreversible excessive growth of autologous tissue, normally associated with more or less distinct loss of specific cell and tissue functions. The consequences of such autonomous and irreversible excessive growth involve considerable impairment of an organism, e.g. a human, and can lead to death.

In view of the dramatic consequences of a cancer or tumor disease, various agents for the treatment of such pathogenic changes have been developed. A large number of such agents are disadvantageous in that they lack specific activity and have a variety of side effects. It is especially the high dosage of particular anti-cancer agents that results in numerous side effects which, despite the dramatic consequences, cause patients to terminate therapy at an early stage.

Another problem in finding anti-tumor agents is that various derivatives, or the original substance and derivatives thereof, exhibit differing effects both in animal models and in humans. For example, various original substances are known which have no or only low anti-tumor effect, whereas derivatives or conversion products thereof may have a significant tumor-inhibiting effect, but also a tumor-promoting effect.

According to Klohe et al. (1985), for example, cis-hydroxyproline lacks the properties required for an effective anti-tumor agent. In view of initial positive tests on the above and other amino acids conducted by the National Cancer Institute during 1933 to 1946, derivatives of proline and hydroxyproline for use as medicaments in cancer therapy have been synthesized during the following years (EP 02 23 850). Due the low effect of CHP (Klohe et al.), it has also been suggested to use a combination of different CHP derivatives, because the latter are said to have a synergistic effect as pharmaceutical agents in tumor treatment (U.S. Pat. No. 6,066,665).

The above-described synergistic effects of the combination preparations were found reproducible only in part, and, in addition, the derivatives developed could only be used at very high concentrations which are accompanied by side effects.

The object of the invention was therefore to provide an agent and a method for cancer therapy based on CHP, which would permit easy, safe and effective application.

A combination of CHP and gemcitabine was found to be highly effective against tumor cells. The invention therefore involves the surprising teaching that a compound, namely, non-derivatized CHP whose anti-tumor properties have been described as insufficient in the prior art, in combination with the chemotherapeutic agent gemcitabine has an effect on cancer cells which, surprisingly, is higher than that of the individual compounds.

CHP in the meaning of the invention includes the cis-isomers of 4-hydroxy-L-proline or salts thereof, which are not CHP derivatives. More specifically, gemcitabine in the meaning of the invention is gemcitabine hydrochloride, i.e., 2′-deoxy-2′,2′-difluorocytidine. For example, the combined agent in the meaning of the invention is such in nature that CHP and gemcitabine together are included in a solution or in a solid, e.g. a tablet, wherein the ratio of CHP and gemcitabine can vary freely. Preferred is a ratio of CHP and gemcitabine in a range of from 1:10,000 to 10,000:1. Depending on the tumor and condition of the patient, the ratio of CHP and gemcitabine can vary within the above range. Of course, said at least two components —CHP and gemcitabine—can also be incorporated together in a solution or solid in such a way that release thereof will proceed in a time-shifted fashion. However, the combined agent in the meaning of the invention may also be constituted of two separate solutions or two separate solids, one solution or solid essentially comprising gemcitabine and the other solution or solid essentially comprising CHP. The two solutions or solids can be associated with a common carrier or with separate carriers. For example, the two solutions and/or the two solids can be present in a capsule as common carrier. Such a formulation of the combined agent of the invention is advantageous in those cases where administration of CHP and administration of gemcitabine are to proceed in a time-shifted manner. That is, the organism is initially contacted with CHP, e.g. by infusion or oral administration, to be contacted with the other component of the combined agent in a time-shifted manner. Of course, it is also possible to provide the combined agent by means of conventional pharmaceutical-technical methods and procedures in such a way that the organism is initially contacted with gemcitabine and subsequently with CHP. Hence, the organism is preferably contacted sequentially with the components of the combined agent. The time period between administration of the two components of the combined agent of the invention or the initial release of CHP or gemcitabine will depend on the age, sex, overall constitution of the patient, the type of tumor, or other parameters which can be determined by the attending physician using prior tests, for example.

Of course, the agent according to the invention may also comprise conventional auxiliaries, preferably carriers, adjuvants and/or vehicles. For example, the carriers can be fillers, diluents, binders, humectants, disintegrants, dissolution retarders, absorption enhancers, wetting agents, adsorbents and/or lubricants. In this event, the combined agent is specifically referred to as drug or pharmaceutical agent.

In a preferred embodiment of the invention the agent comprises one or more additional agents from the group of antiviral, fungicidal or antibacterial agents and/or immunostimulators. Furthermore, the agent may comprise additional chemotherapeutic agents, preferably alitretinoin, aldesleukin (IL-2), altretamine, all-trans-retinoic acid (tretinoin), aminoglutethimide, anagrelide, anastrozole, asparaginase (E. coli), azathioprine, bicalutamide, bleomycin, busulfan, capecitabine, carboplatin, carmustine, chlorambucil, cisplatin, cladribine (2-CDA), cyclophosphamide, cytarabine, dacarbazine, dactinomycin D, daunorubicin (daunomycin), liposomal daunorubicin, dexamethasone, docetaxel, doxorubicin, liposomal doxorubicin, epirubicin, estramustine phosphate, etoposide (VP-16-213), exemestane, floxuridine, 5-fluorouracil, fludarabine, fluoxymesterone, flutamide, gemcitabine, gemtuzmab, goserelin acetate, hydroxyurea, idarubicin, ifosfamide, imatmib mesylate, irinotecan, α-interferon, letrozole, leuprolide acetate, levamisole-HCl, lomustine, megestrol acetate, melphalan (L-phenylalanine mustard), 6-mercaptopurine, methotrexate, methoxsalen (8-MOP), mitomycin C, mitotane, mitoxantrone, nilutamide, nitrogen mustard (mechlorethamine hydrochloride), octreotide, paclitaxel, pegaspargase, pentostatin (2′-deoxycoformycin), plicamycin, porfimer, prednisone, procarbazine, rituximab, streptozotocin, tamoxifen, teniposide (VM-26), 6-thioguanine, thalidomide, thiotepa, topotecan, toremifene, trastuzumab, trimetrexate, vinblastine, vincristine and/or vinorelbine. Partial or complete substitution of gemcitabine by one or more of the above-mentioned agents can also be preferred.

The invention also relates to the use of the agents of the invention in diagnosis, prophylaxis, follow-up, therapy, and/or aftercare of diseases associated with cell growth, cell differentiation and/or cell division.

In a preferred embodiment of the invention, said disease is a tumor, especially a neoplastic tumor, an inflammatory tumor, an abscess, effusion and/or edema. In a particularly preferred fashion the tumor is a solid tumor or a leukemia.

In another preferred embodiment of the invention the agent according to the invention is formulated as a gel, poudrage, powder, tablet, sustained-release tablet, premix, emulsion, brew-up formulation, drops, concentrate, granulate, syrup, pellet, bolus, capsule, aerosol, spray and/or inhalant and/or used in this form. The tablets, coated tablets, capsules, pills and granulates can be provided with conventional coatings and envelopes optionally including opacification agents, and can also be composed such that release of the active substance(s) takes place only or preferably in a particular area of the intestinal tract, optionally in a delayed fashion, to which end polymer substances and waxes can be used as embedding materials.

Preferably, the drugs of the present invention can be used in oral administration in any orally tolerable dosage form, including capsules, tablets and aqueous suspensions and solutions, without being restricted thereto. In case of tablets for oral application, carriers frequently used include lactose and corn starch. Typically, lubricants such as magnesium stearate are also added. For oral administration in the form of capsules, diluents that can be used include lactose and dried corn starch. In oral administration of aqueous suspensions the active substance is combined with emulsifiers and suspending agents.

Also, particular sweeteners and/or flavors and/or coloring agents can be added, if desired.

The active substance(s) can also be present in micro-encapsulated form, optionally with one or more of the above-specified carrier materials.

In addition to the active substance(s), suppositories may include conventional water-soluble or water-insoluble carriers such as polyethylene glycols, fats, e.g. cocoa fat and higher esters (for example, C₁₄ alcohols with C₁₆ fatty acids) or mixtures of these substances.

In addition to the active substance(s), ointments, pastes, creams and gels may include conventional carriers such as animal and vegetable fats, waxes, paraffins, starch, tragacanth, cellulose derivatives, polyethylene glycols, silicones, bentonites, silica, talc and zinc oxide or mixtures of these substances.

In addition to the active substance(s), powders and sprays may include conventional carriers such as lactose, talc, silica, aluminum hydroxide, calcium silicate and polyamide powder or mixtures of these substances. In addition, sprays may include conventional propellants such as chlorofluorohydrocarbons.

In addition to the active substances CHP and gemcitabine, solutions and emulsions may include conventional carriers such as solvents, solubilizers and emulsifiers such as water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substances, suspensions may include conventional carriers such as liquid diluents, e.g. water, ethyl alcohol, propylene glycol, suspending agents, e.g. ethoxylated isostearyl alcohols, polyoxyethylenesorbitol and sorbitan esters, microcrystalline cellulose, aluminum metahydroxide, bentonite, agar, and tragacanth, or mixtures of these substances.

The drugs can be present in the form of a sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e.g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their poly-oxyethylated forms. Such oil solutions or suspensions may also include a long-chain alcohol or a similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the active substances CHP and/or gemcitabine should be present in the above-mentioned pharmaceutical preparations at a concentration of about 0.1 to 99.5 wt.-%, more preferably about 0.5 to 95 wt.-% of the over-all mixture.

In addition to CHP and gemcitabine, the above-mentioned pharmaceutical preparations may include further pharmaceutical active substances. The production of the pharmaceutical preparations specified above proceeds in a usual manner according to well-known methods, e.g. by mixing the active substance(s) with the carrier material(s).

The above-mentioned preparations can be applied in humans and animals on an oral, rectal, parenteral (intravenous, intramuscular, subcutaneous), intracisternal, intravaginal, intraperitoneal route, locally (powders, ointment, drops) and used in the therapy of tumors. Injection solutions, solutions and suspensions for oral therapy, gels, brew-up formulations, emulsions, ointments or drops are possible as suitable preparations. For local therapy, ophthalmic and dermatological formulations, silver and other salts, ear drops, eye ointments, powders or solutions can be used. With animals, ingestion can be effected via feed or drinking water in suitable formulations. Moreover, the drugs or combined agents can be incorporated in other carrier materials such as plastics (plastic chains for local therapy), collagen or bone cement.

In another preferred embodiment of the invention, CHP and/or gemcitabine are incorporated in a pharmaceutical preparation at a concentration of 0.1 to 99.5, preferably 0.5 to 95, and more preferably 20 to 80 wt.-%. That is, CHP and/or gemcitabine are present in the above-specified pharmaceutical preparations, e.g. tablets, pills, granulates and others, at a concentration of preferably 0.1 to 99.5 wt.-% of the overall mixture. Those skilled in the art will be aware of the fact that the amount of active substance, i.e., the amount of an inventive compound combined with the carrier materials to produce a single dosage form, will vary depending on the patient to be treated and on the particular type of administration. Once the condition of a patient has improved, the proportion of active compound in the preparation can be modified so as to obtain a maintenance dose that will inhibit or prevent further growth of the tumor or suppress metastasization and infiltration. Depending on the symptoms, the dose or frequency of administration or both can subsequently be reduced to a level where the improved condition is retained. Once the symptoms have been alleviated to the desired level, the treatment should be terminated. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents in particular can be applied at varying dosages. Application should be effected in such a way that a disease is combated as effectively as possible or the onset of such a disease is prevented by a prophylactic administration. Concentration and type of application can be determined by a person skilled in the art using routine tests. Preferred applications of the compounds of the invention are oral application in the form of powders, tablets, fluid mixture, drops, capsules or the like, rectal application in the form of suppositories, solutions and the like, parenteral application in the form of injections, infusions and solutions, and local application in the form of ointments, pads, dressings, lavages and the like. Contacting with the compounds according to the invention is preferably effected in a prophylactic or therapeutic fashion.

For example, the suitability of the selected form of application, of the dose, application regimen, selection of adjuvant and the like can be determined by taking serum aliquots from the patient, i.e., human or animal, and testing for the presence of cancer cells in the course of the treatment procedure.

Alternatively or concomitantly, the condition of the liver, but also, the amount of T cells or other cells of the immune system can be determined in a conventional manner so as to obtain a general survey on the immunologic constitution of the patient and, in particular, the constitution of organs important to the metabolism. Additionally, the clinical condition of the patient can be observed for the desired effect. Where insufficient anti-tumor effectiveness is achieved, the patient can be subjected to further treatment using the agents of the invention, optionally modified with other well-known medicaments expected to bring about an improvement of the overall constitution. Obviously, it is also possible to modify the carriers or vehicles of the pharmaceutical agent or to vary the route of administration. In addition to oral ingestion, e.g. intramuscular or subcutaneous injections or injections into the blood vessels can be envisaged as another preferred route of therapeutic administration of the compounds according to the invention. At the same time, supply via catheters or surgical tubes can also be used.

In addition to the above-specified concentrations during use of the compounds of the invention, CHP and/or gemcitabine can be employed in a total amount of 0.05 to 500 mg/kg body weight per 24 hours, preferably 5 to 100 mg/kg body weight. Advantageously, this is a therapeutic quantity which is used to prevent or improve the symptoms of a disorder or of a responsive, pathologically physiological condition.

Obviously, the dose will depend on the age, health and weight of the recipient, degree of the disease, type of required simultaneous treatment, frequency of the treatment and type of the desired effects and side-effects. The daily dose of 0.05 to 500 mg/kg body weight can be applied as a single dose or multiple doses in order to furnish the desired results. In particular, pharmaceutical agents are typically used in about 1 to 10 administrations per day, or alternatively or additionally as a continuous infusion. Such administrations can be applied as a chronic or acute therapy. Of course, the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5000 mg/kg. The tablets can also be sustained-release tablets, in which case the number of applications per day is reduced to 1 to 3. The active substance content of sustained-release tablets can be from 3 to 3000 mg. If the active substance—as set forth above—is administered by injection, the host is preferably contacted 1 to 10 times per day with the compounds of the invention or by using continuous infusion, in which case quantities of from 1 to 4000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can determine the optimum dosages required in each case and the type of application of the active substances.

In another particularly preferred embodiment of the invention the pharmaceutical agent is used in a single administration of from 1 to 100, especially from 2 to 50 mg/kg body weight. In the same way as the total amount per day, the amount of a single dose per application can be varied by a person of specialized knowledge in the art. Similarly, the compounds used according to the invention can be employed in veterinary medicine with the above-mentioned single concentrations and formulations together with the feed or feed formulations or drinking water. A single dose preferably includes that amount of active substance which is administered in one application and which normally corresponds to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to the invention is distributed over 2 to 10 applications, preferably 2 to 7, and more preferably 3 to 5 applications. Of course, continuous infusion of the agents according to the invention is also possible.

In a particularly preferred embodiment of the invention, 1 to 2 tablets are administered in each oral application of the compounds of the invention. The tablets according to the invention can be provided with coatings and envelopes well-known to those skilled in the art or can be composed in a way so as to release the active substance(s) only in preferred, particular regions of the host.

In a preferred embodiment the cancerous disease or tumor being treated or prophylactically prevented, or whose reappearance is prevented, is selected from the group of cancerous diseases or tumor diseases of the ear-nose-throat region, of the lungs, mediastinum, gastrointestinal tract, urogenital system, gynecological system, breast, endocrine system, skin, bone and soft-tissue sarcomas, mesotheliomas, melanomas, neoplasms of the central nervous system, cancerous diseases or tumor diseases during infancy, lymphomas, leukemias, paraneoplastic syndromes, metastases with unknown primary tumor (CUP syndrome), peritoneal carcinomatoses, immunosuppression-related malignancies and/or tumor metastases.

More specifically, the tumors may comprise the following types of cancer: adenocarcinoma of breast, prostate and colon; all forms of lung cancer starting in the bronchial tube; bone marrow cancer, melanoma, hepatoma, neuroblastoma; papilloma; apudoma, choristoma, branchioma; malignant carcinoid syndrome; carcinoid heart disease, carcinoma (for example, Walker carcinoma, basal cell carcinoma, squamobasal carcinoma, Brown-Pearce carcinoma, ductal carcinoma, Ehrlich tumor, in situ carcinoma, cancer-2 carcinoma, Merkel cell carcinoma, mucous cancer, non-parvicellular bronchial carcinoma, oat-cell carcinoma, papillary carcinoma, scirrhus carcinoma, bronchio-alveolar carcinoma, bronchial carcinoma, squamous cell carcinoma and transitional cell carcinoma); histiocytic functional disorder; leukemia (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia); malignant histiocytosis, Hodgkin disease, non-Hodgkin lymphoma, solitary plasma cell tumor; reticuloendotheliosis, chondroblastoma; chondroma, chondrosarcoma; fibroma; fibrosarcoma; giant cell tumors; histiocytoma; lipoma; liposarcoma; leukosarcoma; mesothelioma; myxoma; myxosarcoma; osteoma; osteosarcoma; Ewing sarcoma; synovioma; adenofibroma; adenolymphoma; carcinosarcoma, chordoma, craniopharyngioma, dysgerminoma, hamartoma; mesenchymoma; mesonephroma, myosarcoma, ameloblastoma, cementoma; odontoma; teratoma; thymoma, chorioblastoma; adenocarcinoma, adenoma; cholangioma; cholesteatoma; cylindroma; cystadenocarcinoma, cystadenoma; granulosa cell tumor; gynadroblastoma; hidradenoma; islet-cell tumor; Leydig cell tumor; papilloma; Sertoli cell tumor, theca cell tumor, leiomyoma; leiomyosarcoma; myoblastoma; myoma; myosarcoma; rhabdomyoma; rhabdomyosarcoma; ependymoma; ganglioneuroma, glioma; medulloblastoma, meningioma; neurilemmoma; neuroblastoma; neuroepithelioma, neurofibroma, neuroma, paraganglioma, non-chromaffin paraganglioma, angiokeratoma, angiolymphoid hyperplasia with eosinophilia; sclerotizing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phylloides; hemangiosarcoma; lymphangiosarcoma; myxosarcoma, ovarian carcinoma; sarcoma (for example, Ewing sarcoma, experimentally, Kaposi sarcoma and mast cell sarcoma); neoplasms (for example, bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of the head and neck, of the central nervous system, neoplasms of the hearing organ, pelvis, respiratory tract and urogenital tract); neurofibromatosis and cervical squamous cell dysplasia.

In a preferred embodiment the cancerous disease or tumor being treated or prophylactically prevented, or whose reappearance is prevented, is selected from the following group: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy, metastasized tumors comprising brain metastases, lung metastases, liver metastases, bone metastases, pleural and pericardial metastases, and malignant ascites.

In another preferred embodiment the cancerous disease or tumor being treated or prophylactically prevented, or whose reappearance is prevented, is selected from the group comprising cancerous diseases or tumor diseases such as mammary carcinomas, gastrointestinal tumors, including colon carcinomas, stomach carcinomas, large intestine cancer and small intestine cancer, pancreas carcinomas, ovarian carcinomas, liver carcinomas, lung cancer, renal cell carcinomas, multiple myelomas.

Without intending to be limiting, the invention will be explained in more detail with reference to the following example.

Materials and Methods:

Unless otherwise stated, cell lines from the American Type Culture Collection (ATCC, Rockville, Md.) were used and cultured to confluence in a monolayer in RPMI-1640 bicarbonate medium (Seromed, Berlin, Germany) in an incubator (5% H₂O, 37° C.). The cells were examined for mycoplasma contaminations. The medium included 10% heat-inactivated fetal calf serum (Seromed) and 4 mM glutamine. The cells were cultured and passaged according to standard procedures (0.03% trypsin with 0.02% EDTA, 3 times a week) . The cell number was determined using a TOA Sysmex micro-cell counter (TOA, Tokyo, Japan).

Chemicals and Solutions:

Unless otherwise stated, the chemicals were from Sigma (St. Louis, Mo.). The components for testing were used as supplied. CHP was used in the form of a 5 mg/ml stock solution in PBS (phosphate-buffered saline, Dulbecco), and aliquots thereof were frozen at −20° C. Associated components were used in the form of a 2 mg/ml stock solution and frozen at −20° C.

Cell Cycle Analysis and Chemosensitivity Assay:

The cells were obtained by trypsin treatment, washed in PBS, fixed in 70% ethanol at −20° C. for 20 minutes. Subsequently, the cells were washed once more in PBS, transferred into a staining solution including 20 μg/ml propidium iodide (PI), 5 μg/ml RNAse A in 0.05% Monidet P40/PBS and incubated at room temperature overnight. The washed cells were analyzed by means of flow cytometry (Coulter XL-MLC, Coulter, Miami, Fla.), using a Multicycle AV software (Phoenix), to calculate the cell cycle distribution of PI histograms. The percentage of cells in the G1/0 phase (interphase), S phase (DNA synthesis) and G2M phase (mitotic cells) was determined. Apoptotic subGl cells were calculated from PI histograms. All experiments were performed in duplicate.

The chemosensitivity assay was performed in a 96-well microtiter plate with 10⁴ cells per well and 100 μl of medium, and the components to be tested were supplied in a volume of 100 μl. All components were diluted on the microtiter plates, and the plates were incubated under cell culture conditions for 4 days, except for those tests wherein the ratio between application time and reaction was to be determined. The viability of the cells, including the mitochondrial activity, i.e., the ratio of cell survival rate and cell number, was determined using an MTT assay. The tests were performed according to methods well-known to those skilled in the art.

Apoptosis Assay:

Apoptotic or necrotic cells were assayed using annexin V/PI staining experiments according to standard laboratory methods.

Results: Determination of CHP activity in tumor cell lines

TABLE 1
Cell line screening of CHP anti-proliferation activity

| Tissue/Cells | Name | Reaction |
|---|---|---|
| Pancreas cell lines | BxPC3 | −41% |
| | MIAPaCa2 | −45.7% |
| | ASPC1 | −42% |
| | PANC1 | −54% |
| | Capanl | −66% |
| Osteosarcoma tissue | HOS | −68% |
| Prostate tissue | PC3 | resistant |
| Colon cell lines | HT29 | −48% |
| | Colo320DM | −11% |
| | DLD1 | −19.8% |
| | SW620(Ccl227) | −66.4% |
| | SW480 (Ccl228) | −58% |
| | HCT-15 | −10% |
| | Colo205 | −66% |
| Breast tissue | MCF-7 | −43% |
| | T47D | −34% |
| | MDA-MBA231 | resistant |
| Leukemia cells | K562 | −11% |
| Carcinoids | CR01 | −35% |
| | CR02 | −13% |
| Renal cell lines | A498 | −6% |
| | ACHN | resistant |
| Melanoma | A518 | resistant |
| | Me128 | resistant |
| | B607 | −12% |
| | JVSO | −15% |
| Osteoblasts | Calc22 | +8% |
| Fibroblasts | WI38 | resistant |
| | Fib2 (PPH) | resistant |
| | Fib3 (PPH) | −14% |

When using gemcitabine or other functionally analogous compounds in combination with CHP, it was possible to demonstrate that, surprisingly, the values in Table 1 determined in selected cell lines can be significantly improved in a synergistic fashion. Also, it was demonstrated in these tests that the effect of the combined agents of the invention markedly varies from cell line to cell line.

This will be explained in an exemplary fashion with reference to the pancreas carcinoma cell lines. Administration of the combined agent containing CHP and gemcitabine at a ratio of 400 μg/ml:4 μg/ml and release of both compounds at the same time shows an antagonistic effect. That is, the cells live longer. The antagonistic effect of about 15 to 25% was confirmed by varying the concentration of gemcitabine, being 0.25, 0.05 or 1 μg/ml, and varying the concentration of CHP in said combined agent. In particular, these results were found in pancreas carcinoma cell lines, whereas in other cell lines, administration of combined agents releasing CHP and gemcitabine at the same time had an inhibiting effect on cell growth.

Combined agents of the invention sequentially releasing CHP and gemcitabine were also tested. The production of these agents was effected according to pharmaceutical-technical methods well-known to those skilled in the art. Agents were used in cell cultures, which agents initially release CHP, followed by gemcitabine after 24 hours and 48 hours, respectively. The pancreas carcinoma cells initially contacted with CHP, i.e. pre-treated with CHP, were found to show resistance during the subsequent contacting with gemcitabine. Thus, at a higher gemcitabine concentration of more than 0.25 μg/ml, for example, the cells were more resistant by about 5%, and more resistant by up to 20% at lower gemcitabine concentration. That is, administration of combined agents sequentially releasing CHP and gemcitabine in such a way that the cells are initially contacted with CHP and subsequently with gemcitabine results in a reduction of the gemcitabine sensitivity of pancreas tumor cells. It was possible to repeat these results in other cell lines, and it was demonstrated with selected cells that administration of the above-mentioned combined agents resulted in a lower resistance of these cells to gemcitabine. A marked synergistic effect was found when using combined agents releasing gemcitabine first and CHP thereafter. Surprisingly, a synergistic effect occurred in pancreas carcinoma cell lines when the CHP concentration in the combined agent was very low, while an antagonistic effect occurred when the CHP concentration was above 100 μg/ml in the combined agent. Combined agents were used which released 1 μg/ml gemcitabine, followed by CHP after 12, 24 and 48 hours, respectively.

Table 2 shows the effect of the combined agents on PANC-1 cells.

| | | PANC-1 | | BxPC3 |
|---|---|---|---|---|
| | | treated with | | treated with |
| CHP | PANC-1 | agent releasing | BxPC3 | agent releasing |
| (μg/ml) | control | gemcitabine first | control | gemcitabine first |
| 100 | 95.1 ± 2.3 | 103 ± 2.5 | 105.2 ± 5.4 | 98.7 ± 2.0 |
| 50 | 94.8 ± 6.4 | 90.0 ± 8.2 | 99.8 ± 4.3 | 87.3 ± 3.8 |
| 25 | 95.9 ± 5.1 | 88.4 ± 4.0 | 99.9 ± 3.1 | 94.6 ± 3.3 |
| 12.5 | 90.8 ± 4.1 | 88.9 ± 4.3 | 102.6 ± 1.7 | 101.3 ± 4.8 |
| 6.1 | 96.0 ± 6.0 | 91.0 ± 5.9 | 103.5 ± 2.6 | 101.9 ± 8.8 |
| 3 | 98.8 ± 4.0 | 90.3 ± 6.0 | — | — |

That is, sequential treatment of pancreas tumors is particularly promising when the latter are initially contacted with gemcitabine and subsequently with CHP, in which case a low CHP concentration should be selected as set forth above. The agents being used are those including CHP and gemcitabine in carriers and vehicles of varying solubility. Furthermore, kits are used wherein gemcitabine and CHP are provided in separate solutions which are put to use in accordance with instructions contained in the kit. Hence, combined agents are available which, depending on the time-shifted release and on the components or compounds released first or at a later time, exhibit different specificity for different types of tumors. Surprisingly, it was also possible to demonstrate that the combined agent according to the invention modifies the respective cell cycle of the tumor cells. Depending on the combined agent employed and the cells used, there was a modification or loss of the S phase or of the G2M or G1/O phase. Furthermore, as a result of treatment with the combined agent, transition of some cells into apoptotic/necrotic cells was observed. Also, it was possible to demonstrate that varying specificities of the combined agent are achieved when using other chemotherapeutic agents such as oxoplatin or doxorubicin in addition to gemcitabine. This was also demonstrated when using combined agents wherein the gemcitabine portion had been completely replaced by oxoplatin or doxorubicin or another chemotherapeutic agent. Further modification of the specificity of the combined agents was possible by using trans-hydroxyproline instead of the CHP cis-form.

Furthermore, the combined agents disclosed according to the invention were tested clinically on humans. Thus, good results were achieved with the CHP-gemcitabine combination agent and CHP-capecitabine combination agent. Gemcitabine has been approved by the U.S. Food and Drug Administration for initial treatment of patients with locally advanced or metastatic pancreatic adenocarcinomas in 1996. The recommended dosage and the treatment cycle comprise 1 g/m² per week for a period of 7 weeks, followed by one week as a rest period. The subsequent treatment cycles comprise a dose of 1 g/m² per week for three weeks, likewise followed by one week as a phase of rest. However, the above recommended treatment is not free of massive side effects. One typical side effect of gemcitabine administration is e.g. damage of the bone marrow with resulting impairment of the hematopoiesis, so that only few blood cells can come to mature. The consequences of this are anemia, neutropenia and general immunosuppression. The side effects caused by damage of the bone marrow are referred to as myelo-suppression. Other side effects are strong perspiration, diarrhea, fever or influenza-like symptoms, nausea, diarrhea and vomiting, dyspnea, peripheral edemas, hematuria, proteinuria, loss of hair, as well as eczema and reactions at the site of injection.

The above drawbacks can be avoided when using the following combined therapy treatment with gemcitabine and CHP. Progress in the treatment of tumors was determined monthly using computer tomography, clinical laboratory chemistry, determination of tumor markers and physical overall constitution, including hematological examinations. It was found that very good treatment of tumors without appearance of the above-mentioned side effects is possible with said combined therapy.

Regimen of Treatment With the CHP-Gemcitabine Combination Agent

1. Days of treatment 1 to 7: 8 g of CHP daily (intravenous).
2. Treatment on 8 following days: 8 g of intravenous CHP three times a week and 8 g of oral CHP four times a week.
3. If tumor progression is determined, also in terms of the guidelines according to RECIST, additional gemcitabine administration is started.
4. The gemcitabine dose is 1000 mg/m² and is administered intravenously on day 1, 8 and 15.
5. This cycle is repeated on day 29.
6. Concomitantly with gemcitabine infusion, CHP is administered orally, the dose being 4 g.
7. The concomitant administration of CHP is effected on days 3 to 6, 10 to 13, and 17 to 26 (resulting in a 28-day treatment cycle).

A large number of patients suffering from colorectal adenocarcinoma and liver metastases can be treated by means of a combined therapy comprising capecitabine and CHP. The standard treatment for such tumors is gemcitabine administration in the course of a 21-day treatment cycle. The patients are treated for 14 days, followed by a 7-day rest phase. The recommended dose of capecitabine is 2,500 mg/m² per day. The agent is administered orally in two separate doses 30 minutes after each meal. Administration of capecitabine results in a number of side effects as already mentioned for gemcitabine administration (see above). Surprisingly, the combination of capecitabine and CHP results in good efficiency in the treatment of tumors and reduction of side effects compared to the treatment with separate CHP and capecitabine medications. Thus, the combined therapy permits the use of lower doses of capecitabine and shorter treatment cycles of no more than 10 days. The combined therapy is well-tolerated by the patients.

The patients I-K (68 years of age, male) and S-M (76 years of age, male) suffered from histologically determined colorectal adenocarcinoma and liver metastases which were treated using a treatment cycle of combined therapy of capecitabine and CHP according to the following therapeutic regimen:
1. administration of capecitabine for 10 consecutive days (2×3 tablets with 500 mg each), followed by a so-called wash-out period for 10 days, and
2. administration of CHP for 30 consecutive days as an oral solution (dose 8 g each time).

The success of therapy was determined as in the CHP-gemcitabine combination therapy.

Good therapeutic success in tumor treatment was found both in CHP-gemcitabine and CHP-capecitabine combination therapy wherein the chemotherapeutic agents gemcitabine and capecitabine could be used with lower doses and shorter treatment cycles in the presence of CHP (compared to separate administration of the individual pharmaceutical agents). Particularly those side effects appearing in the gastrointestinal tract, such as abdominal pain, diarrhea, including diarrhea and vomiting, vomiting per se, as well as general symptoms of fatigue, as well as stomatitis, anemia and others, were reduced.

1. A combined agent, said agent comprising cis-hydroxyproline (CHP) and gemcitabine or capecitabine.

2. The agent according to claim 1,
further comprising a pharmaceutically acceptable carrier, adjuvant and/or vehicle.

3. The agent according to claim 2,
wherein the carrier is selected from the group comprising consisting of fillers, diluents, binders, humectants, disintegrants, dissolution retarders, absorption enhancers, wetting agents, adsorbents, lubricants and combinations thereof.

4. The agent according to claim 2,
wherein the vehicle is selected from the group consisting of liposomes, siosomes, niosomes and combinations thereof.

5. The agent according to claim 1,
wherein the agent is a gel, poudrage, powder, infusion solution, tablet, sustained-release tablet, premix, a prodrug, emulsion, brew-up formulation, drops, a concentrate, granulate, syrup, pellet, bolus, capsule, aerosol, spray and/or inhalant.

6. The agent according to claim 5,
wherein the CHP and gemcitabine are present in a formulation at a concentration of 0.1 to 99.5, preferably 0.5 to 95, and more preferably 20 to 80 wt. %.

7. The agent according to claim 1,
wherein the CHP and gemcitabine are present in said formulation at a ratio of from 500:1 to 1:500, preferably from 100:1 to 1:100, and more preferably from 50:1 to 1:50.

8. An anti-tumor agent,
comprising a combined agent according to claim 1.

9.-28. (canceled)

29. A method for prophylaxis, therapy, follow-up and/or aftercare of diseases associated with cell growth, cell differentiation and/or cell division comprising
administering to a person benefiting from such prophylaxis, therapy, follow-up and/or aftercare the agent of claim 1 in a prophylaxis, therapy, follow-up and/or aftercare effective amount.

30. The method of claim 29, wherein the disease is a tumor.

31. The method of claim 30, wherein tumor growth, tumor spreading, tumor angiogenesis, tumor invasion, tumor infiltration and/or tumor metastasization is inhibited or prevented.

32. The method of claim 30, wherein the tumor is a neoplastic tumor, inflammatory tumor and/or an abscess, effusion and/or edema.

33. The method of claim 30, wherein the tumor is a solid tumor or leukemia.

34. The method of claim 33, wherein the solid tumor is a tumor of the urogenital tract and/or gastrointestinal tract.

35. The method of claim 30, wherein the tumor is a colon carcinoma, stomach carcinoma, pancreas carcinoma, small intestine carcinoma, ovarian carcinoma, cervical carcinoma, lung carcinoma, prostate carcinoma, mammary carcinoma, renal cell carcinoma, a brain tumor, head-throat tumor, liver carcinoma, and/or a metastase of the above tumors.

36. The method of claim 33, wherein the solid tumor is a mammary, bronchial, colorectal, and/or prostate carcinoma and/or a metastase of the above tumors.

37. The method of claim 34, wherein the tumor of the urogenital tract is a bladder carcinoma and/or a metastase of such tumors.

38. The method of claim 29, wherein said follow-up is monitoring the effectiveness of an anti-tumor treatment.

39. A method for the prophylaxis, prevention, diagnosis, attenuation, therapy, follow-up and/or aftercare of tumor metastasization, tumor invasion, tumor growth, tumor spreading, tumor infiltration and/or tumor angiogenesis comprising
administering to a person benefiting from such prophylaxis, prevention, diagnosis, attenuation, therapy, follow-up and/or aftercare the agent of claim 1 in a prophylaxis, prevention, diagnosis, attenuation, therapy, follow-up and/or aftercare effective amount.

40. The method of claim 39, wherein said follow-up is monitoring the effectiveness of an anti-tumor treatment.

41. The method of claim 29, wherein the agent is used in a combination therapy.

42. The method of claim 39, wherein the agent is used in a combination therapy.

43. The method of claim 42, wherein said combination therapy comprises a chemotherapy, a treatment with cytostatic agents and/or a radiotherapy.

44. The method of claim 41, wherein the combination therapy comprises an adjuvant, biologically specified form of therapy.

45. The method of claim 44, wherein said form of therapy is an immune therapy.

46. A method for increasing sensitivity of tumor cells to cytostatic agents and/or radiation comprising
administering to a person benefiting from the increasing of the sensitivity the agent of claim 1 in a sensitivity of tumor cells to cytostatic agents and/or radiation increasing amount.

47. A method for inhibiting viability, proliferation rate of cells for inducing apoptosis and/or cell cycle arrest comprising
administering to a person benefiting from such inhibiting the agent of claim 1 in an apoptosis and/or cell cycle arrest inducing amount.

48. The method of claims 29, 39, 46 or 47, wherein the agent is administered orally, vaginally, rectally, nasally, subcutaneously, intravenously, intramuscularly, intraperitoneally, regionally and/or topically.

49. The method of claims 29, 39, 46 or 47, wherein the agent is administered in overall amounts of from 0.05 to 1000 mg per kg, preferably from 5 to 450 mg per kg body weight per 24 hours.
